(19) United States
(12) Patent Application Publication
(10) Pub. No.: US 2024/0269243 A1
(43) Pub. Date: Aug. 15, 2024

(54) ALKALINE PHOSPHATASE FOR USE IN THE TREATMENT OF A NEURODEGENERATIVE DISORDER

(71) Applicant: AMRIF B.V., Wageningen (NL)

(72) Inventors: Ronald Sake Oosting, Wageningen (NL); Hiskias Gerrit Keizer, Wageningen (NL); Rudi Brands, Wageningen (NL); Willem Seinen, Wageningen (NL); Herman Feil, Wageningen (NL)

(73) Assignee: AMRIF B.V., Wageningen (NL)

(21) Appl. No.: 18/567,487

(22) PCT Filed: Jun. 8, 2022
(86) PCT No.: PCT/EP2022/065598

§ 371 Date: Dec. 6, 2023

(30) Foreign Application Priority Data
Jun. 9, 2021 (NL) 2028418

(51) Int. Cl. A61K 38/46; A61P 25/28

(52) U.S. Cl. CPC A61K 38/465; A61P 25/28; C12Y 301/03001

(57) ABSTRACT

The treatment of neurodegenerative diseases, i.e. a group of chronic, progressive disorders characterized by the gradual loss of neurons or neuronal function in discrete areas of the central nervous system (CNS). Specifically, alkaline phosphatase (AP) for use in the treatment or prophylaxis of a mammal suffering from or at risk of a neurodegenerative disorder caused by a reduction of Peroxisome proliferator-activated receptor gamma coactivator 1 (PGC1) activity.

The present invention relates to the treatment of neurodegenerative diseases, i.e. a group of chronic, progressive disorders characterized by the gradual loss of neurons or neuronal function in discrete areas of the central nervous system (CNS). Specifically, the present invention relates to alkaline phosphatase (AP) for use in the treatment or prophylaxis of a mammal suffering from or at risk of a neurodegenerative disorder caused by a reduction of Peroxisome proliferator-activated receptor gamma coactivator 1 (PGC1) activity.

Neurodegenerative diseases are a group of chronic, progressive disorders characterized by the gradual loss of neurons in discrete areas of the central nervous system (CNS) and include for example Alzheimer's Disease (AD), Parkinson's Disease (PD) Amyotrophic Lateral Sclerosis (ALS), Multiple Sclerosis (MS) and neurodegeneration following Stroke. Frailty is also included herein, which is an age-related syndrome, and a progressive disorder linked to neurological degradation. Frailty is a comorbidity of diseases and disorders, wherein the body gradually lose their in-built reserves, leaving it vulnerable to dramatic, and sudden changes in health triggered by seemingly small events such as a minor infection or a change in medication or environment, including neurodegeneration. Frailty is related to the ageing process and is generally characterized by issues like reduced muscle strength and fatigue. The mechanism(s) underlying their progressive nature remains unknown but substantial evidence has documented a common inflammatory mechanism in various neurodegenerative diseases. PD is a neurodegenerative disease for which there is currently no treatment. Apart from the well-known motor symptoms as rigidity and tremor, PD is characterized by a multitude of frequently occurring comorbidities (of which most are also seen in frailty) including lowered bone formation, lowered muscle mass, higher gut permeability, worsened intestinal dysbiosis, higher blood brain barrier (BBB) permeability, reduced spatial memory, increased low grade inflammation, and reduced insulin sensitivity.

PD is the second most common neurodegenerative disease after AD and is the most common movement disorder. Currently, about 2% of the population over the age of 60 is affected. Prominent clinical features are motor symptoms such as bradykinesia, tremor, rigidity, and postural instability, and non-motor-related symptoms such as olfactory deficits, autonomic dysfunction, depression, cognitive deficits, and sleep disorders. Like AD, PD is a proteinopathy; it is characterized by the accumulation and aggregation of misfolded α-synuclein. Neuropathological hallmarks are intracellular inclusions containing α-synuclein called Lewy bodies and Lewy neurites and the loss of dopaminergic neurons in the substantia nigra of the midbrain and in other brain regions as well. Loss of dopaminergic neurons is not the only neuropathological alteration in PD, as microglial activation and an increase in astroglia and lymphocyte infiltration also occur. An increase in astroglial cells in post-mortem tissue from the brains of PD patients and an increased number of dystrophic astrocytes have also been reported. Several lines of evidence suggest that inflammatory mediators derived from non-neuronal cells including microglia modulate the progression of neuronal cell death (or loss) in PD.

Frailty and PD share a reduced activity of the PGC/AMPK/Sirt pathway. This pathway orchestrates the primordial stress response of animals, and affects ageing and increases risk of frailty. The PGC/AMPK/Sirt anti-stress pathway is set in motion by non-lethal levels of environmental cellular stress. Most environmental stressors ultimately decrease the energy levels in cells (AMP/ATP ratio), because controlling homeostasis costs more energy under stressful conditions. A decreased AMP/ATP ratio activates the enzyme AMPK, which phosphorylates various target molecules, resulting in an increased NAD/NADH ratio. An increased NAD/NADH ratio induces the activity of a class of proteins called Sirtuins (Sirt). When both AMPK and Sirt are activated under stress, the PGC1 complex is activated. In activated form this complex activates the transcription of genes which code for proteins involved in the anti-stress defence of cells. This includes the formation of anti-oxidative enzymes, the formation of new mitochondria and the tighter closure of gap junctions between cells. Interestingly, under these stressful conditions also uncoupling proteins (UCP) are induced. These further reduce the AMP/ATP levels in cells providing a positive feedback mechanism enabling the cells to react quickly to stressful events by an appropriate anti-stress reaction.

Furthermore, PD and frailty share various common co-morbidities and show a defect in the same biochemical pathway which suggests that a deregulation in this pathway plays a role in the etiology of PD. The involvement of a reduced activity of the PGC/AMPK/Sirt pathway in causing PD is further strengthened by experiments using resveratrol, which is known to activate this specific pathway, wherein resveratrol has the opposite effects as ageing (i.e. frailty) and PD on the comorbidities. The effects on the co-morbidities due to PD or frailty are similar, however these effects are the opposite as the effects seen with exercise or treatment with Resveratrol. To provide further proof that deregulation of the PGC/AMPK/Sirt pathway is involved in PD, an animal model having reduced PGC/AMPK/Sirt pathway was used to research the link with PD. The spontaneously hypertensive rat (SH-rat) is a Wistar-based rat strain having a spontaneous high blood pressure (Okamoto, 1963), and these animals show a considerable decrease in the activity of the PGC/AMPK/Sirt pathway. These animals also show all the PD related co-morbidities. Spontaneously hypertensive rats not only suffer from increased blood pressure they also suffer from reduced bone mass, reduced muscle mass, reduced longevity, altered microflora and insulin resistance. All of these also occur in frailty which is also linked to the PGC1 pathway. Although they do not show rigidity or tremor as expected when PD would develop, they do show a 50% reduction in Th-positive cells in their substantia nigra, and this effect is counteracted by exercise. This suggests that they suffer from a syndrome mimicking the early stages of PD. Together these data indicate that a defective PGC/AMPK/Sirt pathway is to be involved in causing PD and increasing risk of frailty.

Long before motor symptoms occur in PD, the enteric nervous system is affected, and the disease appears to spread from there. These neurons are close to the luminal gut contents of the gut, functionally separated from it by a layer of intestinal cells. As this layer is damaged in PD patients, enteric neurons get exposed to bacterial toxins like LPS. This exposure to bacterial toxins is further enhanced by the fact that Parkinsonian patients suffer from a dysbiosis of its microflora, resulting in more Gram-negative bacteria, producing more LPS. If the primordial PGC/AMPK/Sirt anti-stress pathway is damaged as in PD, this may be sufficient to cause Parkinson's as LPS further inhibits the PGC1/AMPK/Sirt anti-stress pathway. In addition, a study performed in Pink 1-knockout mice showed that symptomless Pink 1-knockout mice became highly afflicted by PD symptoms after having been infected with gram-negative (LPS producing) bacteria. Mechanistically they showed that under these conditions a specific immune reaction was set in motion against dopaminergic neurons, and showed that the Parkinsonian symptoms as induced by these bacteria could be reversed by L-dopa. A dependence on gut bacteria for the development of Parkinsonian symptoms was also observed for alpha-synuclein overexpressing animals. Together this evidence indicates that both genetic and toxin induced types of PD start from the gut where intestinal bacteria strongly contribute to the development of PD.

In PD the PGC/AMPK/Sirt pathway is inhibited by genetic changes or by external toxins. As this pathway controls the intestinal barrier function, this leads to an increased exposure to bacterial toxins. Both these changes activate the (pro-inflammatory) NFkB pathway in the body. A common target for both bacterial toxins and the PGC/AMPK/Sirt pathway is the NFkB pathway: Bacterial toxins, by activating TLR receptors, activate the NFkB pathway, while the PGC/AMPK/Sirt pathway reduces its activity. In PD this will lead to an unbalanced activation of this pathway leading to pro-inflammatory changes in the body which probably contributes to the neurological damage as observed for PD. The key in the search for a cure for neurodegeneration as occurring in PD is finding a target that can be drugged. Directly targeting the NFkB or PGC/AMPK/Sirt pathways by drugs is difficult as both pathways are deeply embedded in the total physiology of the patient, making the selection of drugs with an efficacious and safe spectrum of activities difficult. Therefore, natural ways to restore the natural balance in PD are preferred.

Considering the above, there is a need in the art for a treatment of neurodegenerative diseases characterized by the gradual loss of neurons in discrete areas of the central nervous system (CNS), more preferably there is a need for the treatment of PD and/or frailty and to find a druggable target for treatment providing a long lasting and efficient treatment, and to restore the natural balance in PD and/or frailty in respect to the NFkB or PGC/AMPK/Sirt pathways that are linked to the onset of neurodegenerative diseases, more preferably PD and/or frailty.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by alkaline phosphatase for use in the treatment or prophylaxis of a mammal suffering from or at risk of a neurodegenerative disorder caused by a reduction of Peroxisome proliferator-activated receptor Gamma Coactivator 1 (PGC1) activity as compared to a healthy individual, wherein said treatment comprises administering to said mammal an therapeutically effective amount of alkaline phosphatase that inhibits neurodegeneration or neurodegradation by preventing reduction of PGC1 activity. The administration of alkaline phosphatase (AP) leads to a reduction or prevention of neurodegeneration or neurodegradation, thereby reducing or preventing the neurodegenerative disorder including Parkinson's disease, Alzheimer's disease and frailty.

Experiments show that treatment with alkaline phosphatase protects transgenic C. elegans that suffer from PD (made Parkinsonian by genetic modification) against dopaminergic neurodegeneration. The G2019S LRRK2 C. elegans model was used, as described by YAO, Chen, et al. “LRRK2-mediated neurodegeneration and dysfunction of dopaminergic neurons in a Caenorhabditis elegans model of Parkinson's disease”, Neurobiology of disease, 2010, 40.1: 73-81. In this animal model, the natural LRRK gene of C. elegans was removed and replaced for the human G2019S LRRK2 variant which causes Parkinson's disease in humans. In addition, we show that alkaline phosphatase increases longevity in these Parkinsonian C. elegans worms. The sensitivity of neurons to the mutation causing Parkinson's seems evolutionary conserved in the PGC/AMPK/Sirt pathway and the effect of alkaline phosphatase on longevity is evolutionary conserved because the anti-stress PGC/AMPK/Sirt pathway involved is an evolutionary conserved pathway in mammals. It contains the following targets: AMP/ATP ratio, AMPK, NAD/NADH ratio, SIRT-1, PGC1, and the uncoupling proteins UCPs. All these targets together regulate one general cellular anti-stress response. PGC1 refers to a family of transcriptional coactivators which co-ordinately regulate metabolic pathways and biological processes in a tissue-specific manner. The PGC1 family consists of various family members including at least PGC1alpha and PGC1beta including further subfamily members, of which PGC1alpha4 would be an example. In various organs, often different family members and subtypes are expressed in order to allow precise metabolic control of various organs in a concerted way, as described previously by Léveillé et al, 2020, Molecular Metabolism 34, 72-84.

Furthermore, in an Alzheimer's memory test in transgenic C. elegans CL2355, the therapeutic effect of AP on Alzheimer's Disease (AD) was tested. This experiment makes use of a genetically modified C. elegans strain (CL2355), as was previously described by Yuan Luo et al. in Methods of Behavior Analysis in Neuroscience. 2nd edition, Chapter 16, “Caenorhabditis elegans Model for Initial Screening and Mechanistic Evaluation of Potential New Drugs for Aging and Alzheimer's Disease”. CL2355 expresses human Abeta (Aβ) in all of its neurons in a temperature dependent fashion: Abeta only gets expressed upon shifting the animals from a growth temperature of 16° C., to a growth temperature of 25° C. Because of human Abeta expression these animals develop an impaired memory function which is comparable to AD disease progression in humans also linked to Abeta expression in neurons and memory loss. Results show that treatment of C. elegans worms with AP at 111, 333 and 1000 IU/drop dose dependently, and statistically significantly increased the memory of CL2355 animals compared to buffer treated controls.

Therefore, treatment with AP counteracts the loss of memory in a dose specific manner. The cellular anti-stress response via PGC1 is depicted in FIG. 4. Upon a small ATP deficit (−) or AMP surplus (+) the AMPK is activated resulting in increased ATP production by mitochondria. At higher ATP deficits, CPT1 is activated by AMPK leading to burning of fat of fatty acids releasing from stored fats. If ATP requirement remains high, and CPT1 remains activated, NAD/NADH levels will increase inside cells. This parameter senses an increased oxidation (stress) status, which activates Sirt1. Sirt1 de-acetylates PGC1, AMPK phosphorylates PGC1, and PRMT1 methylates PGC1, thereby activate PGC1 to recruit the nuclear factor PPAR delta. The activated PGC1 and PPAR delta bind to the promotor regions of genes involved in protecting the cells from metabolic and redox stress, leading to their enhanced transcription and provides positive feedback loop on AMP/ATP and further anti-stress response. This anti stress response can be activated by alkaline phosphatase by increasing the AMP/ATP ratio (which activates AMPK) and by detoxifying LPS (which inhibits AMPK activity). This results in an indirect activation of PGC1 which plays an important role in causing Parkinson's disease.

Peroxisome proliferator-activated receptor gamma coactivator 1 (PGC1) acts as a stress sensor in cancer cells and can be activated by nutrient deprivation, exercise, and oxidative damage. It influences mitochondria respiration, reactive oxygen species defence system, and fatty acid metabolism by interacting with specific transcription factors, and in the regulation of both carbohydrate and lipid metabolism. PGC1 is one of the nuclear factors which is activated by AMPK activation and (like AMPK) plays an important role in the anti-stress biochemistry. PGC1 acts as an essential node connecting metabolic regulation, redox control, and inflammatory pathways, and it is an interesting therapeutic target for neurodegenerative diseases such as PD. AMPK is an indirect target for the treatment of PD since it affects the activation of PGC1 in cells. Parkinson's and other neurodegenerative diseases have a defective regulation of PGC1 nuclear factor. Currently, no direct activators of PGC1 exist. AP is used to activate PGC1 via activation and regulation of AMPK, i.e. PGC1 activation by AP is indirect. This enables the body to protect itself in a natural way against direct over-activation of PGC1 e.g. by modulating AMP formation by adenosine kinase and other biochemical pathways.

The mechanism of action of exercise mimetics (which comprise the biological factors that can improve the endurance of humans and animals without the need for training) showed that the same biochemical pathway was triggered by this exercise mimetics as by exercise itself and that the same pathway was probably also important in achieving a healthy prolonged life. The activation of the AMPK/Sirt/PGC1/PPAR complex turned out to be crucial in this. By comparing the biochemistry of longevity and that of Parkinson's, it became clear that PD (as is frailty) is likely caused by a reduction in PGC1 activity. Alkaline phosphatase protects against neurodegeneration in Parkinson's disease in humans via activation of the PGC1 anti-stress pathway and the protective effect of alkaline phosphatase on dopaminergic neurodegeneration is evolutionary conserved. This is further supported by the fact that differentially expressed genes in PD are largely regulated by PGC1, PGC1 expression is lower in PD patients, ageing (which enhances PD and frailty related disorders) leads to reduced expression of PGC1, Exercise, (which inhibits PD neurodegeneration) activates PGC1, and reduced PGC1 in animals results in dopaminergic neurodegeneration. Neurodegeneration in for example PD is caused by PGC1-inactivation, leading to a defective cellular anti-stress response. AP activates cellular anti-stress response, thereby reducing neurodegeneration.

According to a preferred embodiment, the present invention relates to alkaline phosphatase for use in the treatment or prophylaxis of a mammal suffering from or at risk of a neurodegenerative disorder, the wherein the neurodegenerative disorder is selected from the group consisting of Alzheimer's Disease (AD), Parkinson's Disease (PD), frailty related disorders, Amyotrophic Lateral Sclerosis (ALS), Multiple Sclerosis (MS) and neurodegeneration as a result of stroke, preferably Parkinson's Disease, Alzheimer's Disease or frailty related disorders. Examples of neurodegenerative diseases targeted by AP are AD, ALS, neurodegeneration as a result of stroke, Myalgic Encephalomyelitis. The pathological hallmarks of AD in the brain include extracellular amyloid plaques comprising aggregated, cleaved products of the amyloid precursor protein (APP) and intracellular neurofibrillary tangles (NFTs) generated by hyperphosphorylated forms of the microtubule-binding protein tau. Evidence of an inflammatory response in AD includes changes in microglia morphology, from ramified (resting) to amoeboid (active), and astrogliosis (manifested by an increase in the number, size, and motility of astrocytes) surrounding the senile plaques. Although the exact pathophysiological mechanisms underlying neurodegeneration in ALS remain uncertain, a common pathological hallmark is the presence of ubiquitin-immunoreactive cytoplasmic inclusions in degenerating neurons, followed by a strong inflammatory reaction. Prominent neuroinflammation can be readily observed in pathologically affected areas of the CNS and in spinal cords. Typically, inflammation in ALS is characterized by gliosis and the accumulation of large numbers of activated microglia and astrocytes. MS is an autoimmune disease that is characterized by inflammation, demyelination, and axon degeneration in the CNS, more specifically by infiltration of lymphocytes and antibody-producing plasma cells into the perivascular region of the brain and spinal cord white matter, an increase in microglia and astrocytes, and demyelination. Frailty is related to the ageing process, wherein the body gradually lose their in-built reserves, leaving it vulnerable to dramatic, sudden changes in health triggered by seemingly small events such as a minor infection or a change in medication or environment, including neurodegeneration. Frailty is generally characterized by issues like reduced muscle strength and fatigue.

According to a preferred embodiment, the present invention relates to alkaline phosphatase for use in the treatment or prophylaxis of a mammal, preferably a human, suffering from or at risk of a neurodegenerative disorder, wherein said preventing reduction of PGC1 activity comprises an increase in the activation of adenosine monophosphate-activated protein kinase (AMPK) by alkaline phosphatase to promote an anti-stress response Alkaline phosphatase activates AMPK by inducing AMP production once stressed adjacent cells release ATP to the cellular environment. This ATP is metabolized into adenosine by extracellular alkaline phosphatase. Consequently, the target cells take up the adenosine as formed by alkaline phosphatase and phosphorylate it inside the cell into AMP which activates AMPK. Alternatively, the adenosine as formed from ATP by alkaline phosphatase activates purinergic surface receptors on target cells which can also activate AMPK. Furthermore, AP also detoxifies LPS which is a proinflammatory molecule that inhibits AMPK activity in target cells.

According to a preferred embodiment, the present invention relates to alkaline phosphatase for use in the treatment or prophylaxis of a mammal suffering from or at risk of a neurodegenerative disorder, wherein said therapeutically effective amount is 6 to 1350 U/day/kg, preferably 25 to 750 U/day/kg, more preferably 50 to 500 U/day/kg alkaline phosphatase. AP activity is defined in (Glycine) Units/ml as disclosed in Bergmeyer, H. U. (1974) Methods of Enzymatic Analysis, 2nd edition, p 496, Academic Press, New York. 1 unit is the amount of enzyme capable to convert 1 μM of para-nitro-phenol phosphate (PNPP) per minute at 25° C. and pH 9.6 (glycine buffer). Experiments with AP were performed in C. elegans and show that AP significantly extends lifespan at 1000 U in comparison to the untreated group. The average lifespan of untreated and treated nematodes at 1000 U of AP is found to be 14.93+0.52 days and 17.37+0.92 days, respectively. When the AP dose was lowered to 200 U, the effect of AP was also observed, although less pronounced. These results, when extrapolated to mammals, more specifically humans, results in an expected therapeutically effective amount of AP of between 500 to 100,000 U per day per person, preferably 5000 to 30,000 (on average ˜75 kg bodyweight), or a therapeutically effective amount of AP of 6.7 to 1333.3 U/day/kg for humans, preferably 66.7 to 400 U/day/kg.

According to a preferred embodiment, the present invention relates to alkaline phosphatase for use in the treatment or prophylaxis of a mammal suffering from or at risk of a neurodegenerative disorder, wherein the alkaline phosphatase is a tissue specific ecto-phosphatase selected from the group consisting of intestinal AP (IAP), placental ALP (PALP) and liver AP (LAP), preferably IAP or PALP. Intestinal alkaline phosphatase (IAP) can be used to treat PD. A tissue non specific ectophosphatase may also be suitable for use in the treatment of present invention. PD starts with a reduced activity of the PGC/AMPK/Sirt partway. The reduced activity of this pathway can have a genetic cause, but also toxins such as LPS reduce the activity of this pathway. SH-rats, which show a decreased PGC/AMPK/Sirt pathway, also show strongly reduced levels intestinal alkaline phosphatase. Activators of the PGC/AMPK/Sirt pathway, including oleic acid and curcumin, increase the expression of IAP in the gut. IAP has a profound effect on intestinal tract microflora, and a reduction in alkaline phosphatase activity result in the dysbiosis of the microflora in PD. A reduction in the activity of the PGC/AMPK/Sirt pathway as observed in PD leads to a downregulation of tight junctions. In the intestinal this leads to increased exposure to bacterial toxins, including LPS, and consequently to immune activation by exposure of TLR receptors in the intestinal immune system. In the brain this will lead to an increased permeability of the blood brain barrier to toxins and circulating immune cells causing inflammation. Increased exposure of neuronal tissue to bacterial toxins leads to an increase in expression of alpha synuclein. This expression promotes the leakiness of the Blood brain barrier as induced by LPS and activation of the immune system to target the brain in PD.

According to a preferred embodiment, the present invention relates to alkaline phosphatase for use in the treatment or prophylaxis of a mammal suffering from or at risk of a neurodegenerative disorder, wherein Said treatment comprises intravenous, parenteral or oral administration, preferably oral administration. For oral administration IAP is preferred and for parenteral use PALP is more preferred. Oral alkaline phosphatase may be a treatment for PD, because such a treatment will promote the growth of commensal bacteria in the gut, promote the closure of intestinal barriers and detoxify various bacterial toxins including LPS and ATP. Consequently, AP would normalize the gut intestinal flora, close the leaky gap-junctions in the gut and reduce the neuro-inflammation induced by these changes as well as by activating efferent nerve endings of the Vagal nerve, thereby preventing the onset of PD. Additional observations support the invention that orally dosed IAP can be used for the treatment in PD. Research indicated that the adaptive immune response leading to PD is controlled by Th17 cells. Th17 cells can only mature in the gut in the presence of a suitable pro-inflammatory gut microflora and this maturation depends on luminal ATP in the intestines. As IAP rapidly dephosphorylates luminal ATP, it also to inhibit Th17 cell activation. Orally dosed IAP can thus play an important factor in steering the adaptive immune and prevent the onset of PD.

Furthermore, as indicated above the PGC/AMPK/Sirt pathway is affected in PD. This pathway is also responsible for increasing longevity. Our results indicate that IAP when dosed to C. elegans significantly prolonged their lifespan, suggesting that it can activate the PGC/AMPK/Sirt pathway in the whole body by just acting inside the gut. Oral AP improves symptoms of metabolic syndrome which is known to be an important risk factor for PD. It was shown that the gut microflora of PD patients modified by probiotics has positive effects on PD symptoms, and insulin sensitivity and plasma triglycerides. However, probiotics often have limited effects on the intestinal microflora. The effect of treatment with AP is preferred, since it is long-lasting and more natural as the normal microflora is likely to be restored whereas with dosing of probiotics it will depend on the exact strain or mixture of strains of bacteria dosed.

According to a preferred embodiment, the present invention relates to alkaline phosphatase for use in the treatment or prophylaxis of a mammal suffering from or at risk of a neurodegenerative disorder, wherein the alkaline phosphatase is a recombinant alkaline phosphatase, preferably a recombinant mammalian alkaline phosphatase, more preferably a human recombinant alkaline phosphatase. Preferably the phosphatase used in the composition of present invention is compatible with the foreseen therapeutic intervention that it is to support, e.g. the treatment of a human being using the composition of present invention comprising a recombinant human alkaline phosphatase. However also other combinations may be used, for instance the treatment of a human being using the composition of present invention comprising a non-human native or non-human recombinant alternative alkaline phosphatase, like e.g. bovine or porcine intestine derived alkaline phosphatases.

According to a preferred embodiment, the present invention relates to alkaline phosphatase for use in the treatment or prophylaxis of a mammal suffering from or at risk of a neurodegenerative disorder, wherein the alkaline phosphatase is one or more selected from the group consisting of a biological active fragment or derivative of alkaline phosphatase, synthetic alkaline phosphatase derivative, and a small chemo-pharmaceutical molecule exerting functional alkaline phosphatase activity, preferably a biological active fragment or derivative of alkaline phosphatase. The biological active fragment or derivative of alkaline phosphatase enables the restoration and protect the functional properties and integrity of the blood brain barrier, the lymphatic system in the brain, and the functional neuro-supportive properties of the microglial and astroglial system in the central nervous system.

According to a preferred embodiment, the present invention relates to alkaline phosphatase for use in the treatment or prophylaxis of a mammal suffering from or at risk of a neurodegenerative disorder, wherein said treatment or prophylaxis comprises the delay of onset, or attenuated progression or prevention of progression of said neurodegenerative disorder. A sustained inflammatory reaction is present in acute onset (e.g. stroke) and chronic (e.g. AD, PD, MS) neurodegenerative disorders. Each of these disorders is distinguished by a disease-specific mechanism for induction of inflammatory responses. The distinct pathways for the induction of inflammation and the specific anatomical locations at which these processes occur are likely determinants of the specific pathological features of each neurodegenerative disease. Remarkably, however, once induced there appears to be considerable convergence in the mechanisms that lead to amplification of inflammatory responses, neurotoxicity, and neuronal death. Activation of innate immune cells in the CNS, such as microglia and astrocytes, is one of the universal components of neuroinflammation. In the diseased CNS, interactions between damaged neurons and dysregulated, overactivated microglia create a vicious self-propagating cycle causing uncontrolled, prolonged inflammation that drives the chronic progression of neurodegenerative diseases.

According to a preferred embodiment, the present invention relates to alkaline phosphatase for use in the treatment or prophylaxis of a mammal suffering from or at risk of a neurodegenerative disorder, wherein said treatment comprises attenuation of the inflammatory response of a mammal suffering neurodegenerative disorder. In many inflammatory conditions it is shown that AP safely and effectively target inflammatory mechanisms, also those that contribute to the pathogenesis of various neurodegenerative disorders. As neurodegenerative disorders are chronic diseases, it is likely that their prevention and treatment will require long-term therapy, imposing a corresponding requirement for a high level of safety. In clinical studies performed with AP, no signs of adverse activity have been observed in patients. Also, in repeated dose toxicity studies with various animal species, that are immune-tolerant for this protein, the animals tolerated high dose daily intravenous injections with AP. Therefore, we expect that AP can be safely applied in patients with neurodegenerative disorders.

According to a preferred embodiment, the present invention relates to alkaline phosphatase for use in the treatment or prophylaxis of a mammal suffering from or at risk of a neurodegenerative disorder, wherein said treatment or prophylaxis comprises promoting the activation of anti-inflammatory cytokines selected from the group consisting of IL-1, IL-4, IL-6, IL-10, IL-11, and IL-13, preferably IL-11. Like mononuclear white blood cells, non-neuronal microglial cells perform analogous functions in immunomodulation to those of macrophages in circulation and are activated and de-activated by pro- and anti-inflammatory factors. LPS- and ischemia-induced inflammatory conditions are resolved by AP activity. Preliminary in-vitro studies on mouse BV2 microglial cells demonstrate that AP skews the activation profile of ATP-stimulated microglia in favour of the M2 anti-inflammatory phenotype, as deduced from a selective increase in the expression of the anti-inflammatory M2 marker cytokine IL-10 (results not shown). AP also attenuates the M1 pro-inflammatory activation of LPS-stimulated microglia in terms of decreased mRNA expression levels of the pro-inflammatory M1 marker cytokines TNF-α, IL-6, and IL-1β.

According to a further aspect, the present invention relates to a method for inhibiting neurodegradation, by preventing reduction of PGC1 activity of a mammal comprising administering to said mammal a therapeutically effective amount of an alkaline phosphatase, as defined above.

According to another aspect, the present invention relates to the use of an alkaline phosphatase as disclosed herein for the preparation of a medicament for the prophylaxis of a mammal at risk of a neurodegenerative disorder caused by a reduction of PGC1 activity as compared to a healthy individual.

The present invention will be further detailed in the following examples and figures wherein:

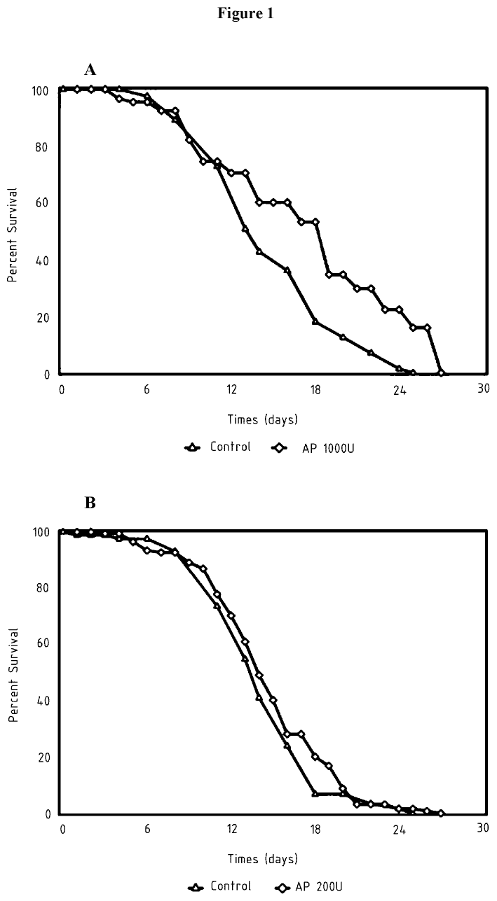

FIG. 1: shows the survival curves of transgenic LRRK2-G2019S C. elegans. Longevity can be increased in C. elegans mutants carry the human G2019S LRRK2 mutation by externally added alkaline phosphatase increases (200 U (A) and 1000 U (B) of AP). Based on this observation it is expected that that under standard living conditions, alkaline phosphatase increases the life span of worms and mammals, since the effect of alkaline phosphatase on longevity is evolutionary preserved (similar pathways, similar mechanism).

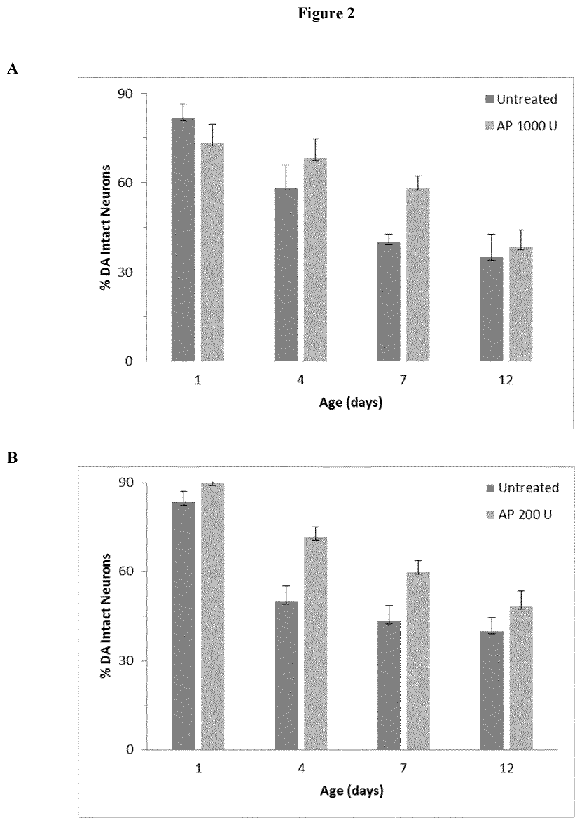

FIG. 2: shows the percentage of dopaminergic (DA) neuronal survival (% intact DA neurons) in transgenic LRRK2-G2019S C. elegans over time that were grown in the absence and presence of 1000 U (A) or 200 U (B) of AP on adult day 1, 4, 7, and 12.

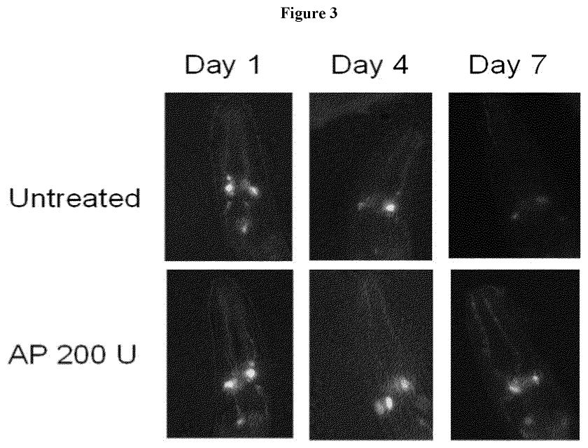

FIG. 3: shows fluorescence microscopy of GFP-tagged DA neurons in transgenic LRRK2-G2019S C. elegans grown in the absence and presence of 200 U AP on adult day 1, 4, and 7.

FIG. 4: shows the regulation of the cellular anti-stress response via PGC1. Regulators in the oval are involved in the transcriptional stress response of cells, wherein PGC1 is in the very centre. Upon a small ATP deficit (−) or AMP surplus (+) the AMPK is activated resulting in increased ATP production by mitochondria. At higher ATP deficits, CPT1 is activated by AMPK leading to burning of fat of fatty acids releasing from stored fats. If ATP requirement remains high, and CPT1 remains activated, NAD/NADH levels will increase inside cells. This parameter senses an increased oxidation (stress) status, which activates Sirt1. Sirt1 de-acetylates PGC1, AMPK phosphorylates PGC1, and PRMT1 methylates PGC1, thereby activate PGC1 to recruit the nuclear factor PPAR delta. The activated PGC1 and PPAR delta bind to the promotor regions of genes involved in protecting the cells from metabolic and redox stress, leading to their enhanced transcription and provides positive feedback loop on AMP/ATP and further anti-stress response. This anti stress response can be activated by alkaline phosphatase by increasing the AMP/ATP ratio (which activates AMPK) and by detoxifying LPS (which inhibits AMPK activity). This results in an indirect activation of PGC1 which plays an important role in causing Parkinson's disease.

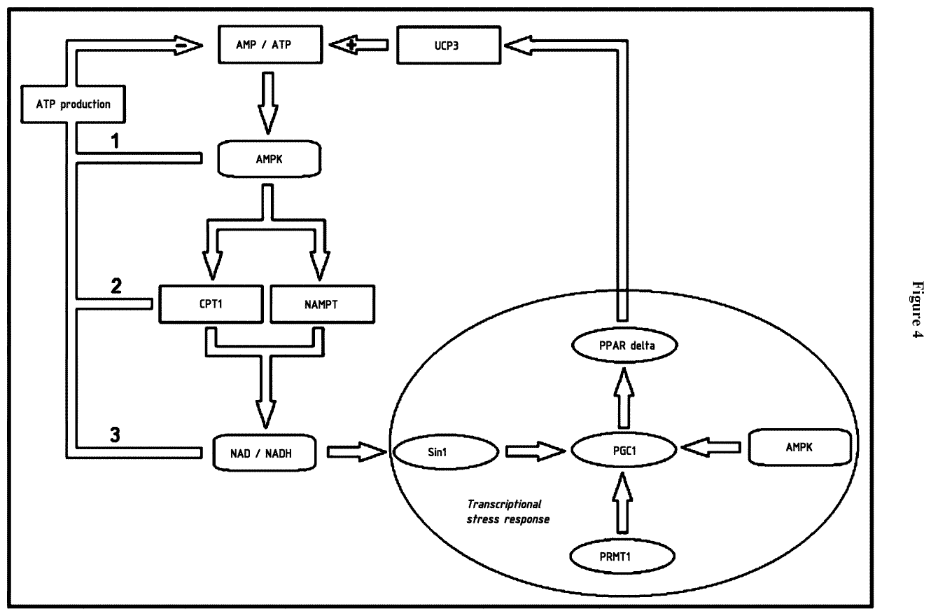

FIG. 5: shows results of the memory test of trained C. elegans CL2122 (control group) and CL2355 (Alzheimer's disease model group) after treatment with different dose AP or Metformin (positive control). Memory is expressed as chemotaxis index in view of the respective treatments in CL2122 and CL2355 animals. The chemotaxis index is calculated as the number of animals in the butanone spot minus the number of animals in the ethanol spot divided by the total number of animals as determined in the memory test (see example 2 for details). Memory of Abeta-expressing worms, i.e. CL2355 are worms that express the human Abeta leading to severe memory loss, was close to zero. CL2122 do not express Abeta and serve as control group. AP shows a dose response in view of the improvement of memory in C. elegans CL2355. Treatment of worms with AP at 111, 333 and 1000 IU/drop dose dependently, and statistically significantly increased the memory of CL2355 animals compared to buffer treated controls. Also, the metformin treated animals showed a statistically significant increase in memory in this test.

### EXAMPLES

### Example 1—In Vivo Effect of AP on Lifespan and Neurodegeneration of Transgenic LRRK2-G2019S C. elegans

Here were test for the effect of AP in the protection of dopaminergic neurons in animal models of PD. In this study, transgenic LRRK2-G2019S C. elegans was used to assess the effect of AP on lifespan and neurodegeneration. The G2019S LRRK2 C. elegans model was used, as described by YAO, Chen, et al. “LRRK2-mediated neurodegeneration and dysfunction of dopaminergic neurons in a Caenorhabditis elegans model of Parkinson's disease”, Neurobiology of disease, 2010, 40.1: 73-81. In this animal model, the natural LRRK gene of C. elegans was removed and replaced for the human G2019S LRRK2 variant which causes Parkinson's disease in humans. The C. elegans comprises orthologue genes of AMPK, Sirt1 and PGC1; AAK, Sir2.1 and MDT15/NHR49 complex, respectively. The AAK and Sir2.1 (and also AMPK and Sirt1 of course) are enzymes that change other proteins in activity. This eventually leads to the activation of protein complexes that trigger DNA transcription of anti-stress genes in the nucleus of the cell. This transgenic C. elegans were used to assess the effect of AP on the survival of dopaminergic neurons.

The following experiments were performed; A lifespan experiment is performed to determine the efficacy of AP for lifespan extension in transgenic LRRK2-G2019S C. elegans grown on live bacterial lawns. Furthermore, a neurodegeneration experiment was to test the efficacy of AP for the protection of dopaminergic neurons from degeneration during ageing and frailty related symptoms in transgenic C. elegans grown on live bacterial lawns. The above two experiments were subsequently repeated to confirm the effects of AP on both lifespan extension and neuroprotection in transgenic LRRK2-G2019S C. elegans. In addition, microscopic images of dopaminergic neurons were taken during the neurodegeneration experiment.

Nematode growth medium and agar plates are prepared as follows; Agar solution was made by dissolving 3 g NaCl, 17 g agar, and 2.5 g peptone in 975 ml double distilled water using a stir bar and stir plate. The agar solution was autoclaved on a liquid cycle for 30 minutes at 121° C., along with 500 ml distilled water and the dispenser tubing. The agar solution was allowed to cool to 75° C. while being stirred on the stir plate. Using sterile techniques, 1 ml of 1M MgSO₄, 1 ml of 1M CaCl₂, 1 ml of 5 mg/ml cholesterol, and 1 ml of 1M KPO₄ were added to the agar solution. For 35 mm plates, 1 ml of FUdR (75 μM stock solution) was also added to the agar solution. For 35 mm plates, 4 ml of FUdR and agar solution was dispensed into each plate, providing a FUdR+ plate. For 60 mm plates, 8 ml of agar solution was dispensed into each plate. Plates were stored at room temperature for 2 days, covered by plastic trays sterilized with 70% EtOH, to allow the agar to set completely.

E. coli OP50 solution was prepared by using a sterile pipet tip to harvest E. coli OP50 from previously seeded agar plate by scraping the pipet tip across the bacteria lawn. The pipet tip was incubated in one liter of LB broth overnight at 37° C. The E. coli solution was stored in a 4° C. cold room until agar plate seeding. The E. coli OP50 solution was used to seed plates by pipetting 50 μl onto the 35 mm plates, and 150 μl onto the 60 mm plates. Seeded plates were stored at room temperature, covered by plastic trays sterilized with 70% EtOH, until bacteria lawns developed. Seeded plates with E. coli lawns were stored in a 4° C. cold room until used.

An Alkaline Phosphatase (AP) solution was prepared by dissolving AP in a buffer (AP Buffer) consisted of 20 mM Tris (pH 7.8) with 5 mM MgCl₂ and 0.1 mM ZnCl₂, resulting in to a concentration of 50,000 units per ml. The solution was separated into 100 μl aliquots and stored at 4° C.

Prior to starting either assay, transgenic LRRK2-G2019S C. elegans were age-synchronized to initiate development from eggs following alkaline bleaching, the C. elegan sample was age matched. To do this, 10 μl of bleaching solution (25 μl of 5M NaOH, 100 μl of 8% bleach, and 375 μl of double distilled water) was pipetted onto a 60 mm plate away from the bacteria lawn. Fifteen C. elegans in the L4 stage were selected from the breeding population, and using a sterile platinum wire were placed in the bleaching solution on the 60 mm plate. Additional bleaching solution was added as the solution evaporated from the plate until all eggs had been released. This procedure was repeated at a second spot on the same 60 mm plate. Eggs were given 2 days to hatch and grow, and 30 hatched C. elegans were transferred to each 35 mm FUdR+ plate at the start of each assay.

35 mm FUdR+ plates were divided into 4 μl AP (200 U) solution treatment, 20 μl AP (1000 U) solution treatment, and control groups. Treatment group plates were treated by pipetting their respective volume of AP solution onto the bacteria lawn of the plate directly prior to when the C. elegans were transferred. Solution was spread across the lawn by tilting the plate, allowing the solution to coat the entire lawn. Separate control groups were maintained alongside each treatment group, and were scored and transferred on the same days. Every plate was given 15 min to dry after solutions were applied prior to when the C. elegans were transferred.

### Lifespan Assay

Each trial consisted of 3 treatment plates, either 20 μl AP or 4 μl AP, and 3 control plates. C. elegans were scored and transferred daily until egg laying stopped. They were scored every 1 to 3 days, and transferred every 2 to 4 days after egg laying stopped. C. elegans were scored as dead if they did not move in response to being lightly touched with a sterile platinum wire, or censored if they were alive and unable to be transferred, such as being trapped under agar, or appeared to die of unnatural causes, such as an egg hatching within their body. This continued until no worms remained.

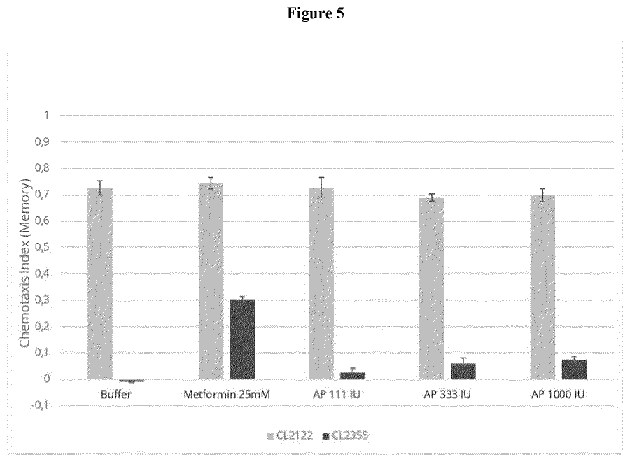

In this study, two independent groups of transgenic LRRK2-G2019S C. elegans were treated with either 200 U or 1000 U of AP. Based on the results a survival curve was obtained of transgenic LRRK2-G2019S C. elegans grown in the absence and presence of 1000 U of AP (FIG. 1A) or 200 U of AP (FIG. 1B). It was confirmed that AP significantly extends lifespan at 1000 U. The average lifespan of untreated and treated nematodes at 1000 U of AP is found to be 14.93+0.52 days and 17.37+0.92 days, respectively. When the AP dose was lowered to 200 U, the effect of AP was markedly reduced. The average lifespan of untreated and treated nematodes at 200 U of AP is found to be 14.34+0.50 days and 14.64+0.38 days, respectively.

AP activity is defined in (Glycine) Units/ml as disclosed in Bergmeyer, H. U. (1974) Methods of Enzymatic Analysis, 2nd edition, p 496, Academic Press, New York. These results, when extrapolated to mammals, more specifically humans, results in an expected therapeutically effective amount of AP of between 500 to 100,000 U per day per person, preferably 5000 to 30,000 (on average ˜75 kg bodyweight), or a therapeutically effective amount of AP of 6.7 to 1333.3 U/day/kg for humans, preferably 66.7 to 400 U/day/kg.

### Neurodegeneration Assay

Each trial began with 4 treatment plates, either 20 μl AP or 4 μl AP, and 4 control plates. Living C. elegans were transferred daily until egg laying stopped, and every 2 to 4 days afterwards. The number of living dopaminergic (DA) neurons in the head of 10 worms from each group were counted using a florescence microscope on the first or second day of the experiment, and every 3 to 4 days afterwards until samples from 4 different days were taken. If there were less than 10 living worms on the last day, the living worms were scored and the remainder of the 10 were marked as dead and containing no living neurons. To score neurons, an adhesive plastic binder reinforcement ring was adhered to the surface of a glass microscope slide. 2 μl of 1× mounting solution (10× stock solution: 10 mg of 1% tricaine and 1 mg of 0.1% tetramisole dissolved in water), and 4 μl of double distilled water was pipetted onto the glass slide in the centre of the adhesive plastic ring. 10 C. elegans from the treatment group sample were transferred into the solution on the slide. A slide cover was placed on the slide, and once the mounting solution had immobilized the C. elegans, the slide cover was glued to the slide using super glue. If the mounting solution failed to immobilize the C. elegans within 5 minutes, 2 μl of additional mounting solution was added. Additional mounting solution was added every 5 minutes until C. elegans were successfully immobilized.

The GFP-tagged dopaminergic neurons in the live worms were observed under a fluorescence microscope. Using the florescence microscope, the number of healthy DA neurons were counted. The number of missing and unhealthy neurons was also recorded. Neurons were considered unhealthy if the cell body appeared shriveled, or the axon appeared broken or beaded. This was repeated for the control sample as well. The dopaminergic neurons were scored for signs of degeneration due to either missed and shrunk cell bodies or broken neurites. The number of neurons that remain intact were plotted against the age of the C. elegans in each group (FIG. 2).

Two independent groups of transgenic LRRK2-G2019S C. elegans treated with either 200 U (FIG. 2A) or 1000 U (FIG. 2B) of AP, showed that AP protected dopaminergic neurons from age-dependent degeneration at both 200 U and 1000 U. AP significantly enhanced dopaminergic neuronal survival measured on adult day 4 (p<0.001) and day 7 (p<0.001) at 200 U AP and on adult day 7 at 1000 U AP (p<0.001). At 7 days after birth (comparable to an age of about 50 years in humans), about 50% of the dopaminergic neurons are damaged in these mutant C. elegans animals, which is comparable to early onset of Parkinson's disease in C. elegans, and comparable to the situation as in Parkinson's patients.

Consistent with the DA neuron counting as shown above, AP attenuated age-dependent diminution of GFP signals in DA neurons of LRRK2-G2019S C. elegans, indicative of enhanced DA neuron survival following treatment of AP. Representative images of DA neurons in LRRK2-G2019S C. elegans treated with 200 U AP on adult day 1, 4, and 7 were shown in FIG. 3.

### DISCUSSION

Experiments show that the lifespan of the transgenic C. elegans is extended by addition of AP at 1000 U, supporting the finding that AP promotes healthy ageing and reduces frailty related disorders in the C. elegans model for PD. These data support the finding that AP can be used to activate our anti-stress biochemistry which also regulates longevity and anti-stress pathway such that neuroprotection should occur. Furthermore, experiments show that AP provides protection of dopaminergic neurons from age-dependent degeneration in transgenic LRRK2-G2019S C. elegans. This in vivo effect was statistically significant at both 200 U/plate and 1000 U/plate. The protective effects of AP on dopaminergic neurons in the same neurodegeneration model is comparable or better than that of drugs (GW5074, Sorafenib and AdoCbl) reported earlier to be effective in this C. elegans model for PD. These results have confirmed the beneficial effects of AP on lifespan and neurodegeneration in transgenic LRRK2-G2019S C. elegans, a model of Parkinson's disease. AP at 1000 U was shown to extend lifespan, while AP at 200 U and 1000 U was shown to rescue dopaminergic neurodegeneration occurring in transgenic LRRK2-G2019S C. elegans. It seems that high doses of AP are required for lifespan extension, while neuroprotective effects can be detected at relatively lower doses.

### Example 2—In Vivo Effect of AP on Alzheimer's Memory Test in C. elegans Transgenic CL2355

In a further experiment, the therapeutic effect of AP on Alzheimer's Disease (AD) was tested. This experiment makes use of a genetically modified C. elegans strain (CL2355), as was previously described by Yuan Luo et al. in Methods of Behavior Analysis in Neuroscience. 2nd edition, Chapter 16, “Caenorhabditis elegans Model for Initial Screening and Mechanistic Evaluation of Potential New Drugs for Aging and Alzheimer's Disease”. CL2355 expresses human Abeta (Aβ) in all of its neurons in a temperature dependent fashion: Abeta only gets expressed upon shifting the animals from a growth temperature of 16° C., to a growth temperature of 25° C. Because of human Abeta expression these animals develop an impaired memory function which is comparable to AD disease progression in humans also linked to Abeta expression in neurons and memory loss. The C. elegans strain CL2122 is used as control strain. This strain has the same genetic background as CL2355 but does not express any Abeta upon temperature shifting.

The effect of alkaline phosphatase (AP) on the Abeta-induced memory loss in CL2355 C. elegans animals is determined. Metformin, which is a AMPK activator and is known to enhance the memory of Abeta expressing CL2355 animals, as disclosed by Waqar Ahmand et al. 2017, Molecular Neurobiology, 54, 5427-5439, was used as a positive control. After treatment with the test compounds, either AP or Metformin, the animals are starved for an hour. Thereafter in order to train them, they are placed on regular NGM agar plates for one hour in the presence (training condition) of absence of butanone (nontrained). After this hour they are starved again for an hour and are ready to be tested for memory retention.

### Preparing Exposure-Plates for Test Compounds

NGM agar plates to be used for exposure of animals to test compounds, were prepared 4 days before the memory test was conducted and stored at 4° C. in the dark. To prepare test compound containing plates, on each 10 cm diameter NGM plate, 10 drops of 50 μl of a 3-times concentrated standard OP50 bacterial culture was pipetted. After these drops had dried, test compounds were added.

For dosing negative controls, 20 μl of buffer was used to dissolve AP. The buffer consists of 20 mM of Tris buffer with 5 mM of MgCl23 and 0.1 Mm of ZnCl2. AP was tested at three different dose levels being 2.2, 6.6 and 20 μl of a solution of AP (50.000 IU/ml), corresponding to 111, 333 and 1000 IU of AP. Metformin was dosed in a solution of 2.5 μl of a 1.5 mM aqueous solution.

Exposure of C. elegans to Test Compounds

About 1000 synchronized animals in the L4 stage (grown at 16° C.) were exposed to test compound for 24 hours at 16° C. by pipetting them on plates with test compound as described above. After these 24 hours, animals were placed on fresh plates and test compounds and incubated for another 24 hours at 25° C. and memory retention was determined by performing a memory retention test as described below.

### Memory Retention Test, Chemotaxis Index

To prepare NGM agar plates for the memory test, two drops of 1 μl of 1M of Sodium Azide are pipetted on 10 cm diameter agar plates, about 6 cm apart from each other in the middle of the plate. The presence of Azide paralyses the animals which come close to these spots. Thereafter, on top of one of the Azide drops, 1 μl of ethanol is pipetted. On top of the other azide drop, 1 μl of a 10% butanone dissolution in ethanol. Now the plates are ready to be used for the memory test.

Subsequently, about 300 worms to be tested are released at the origin, i.e. when taking the middle of the plate, drop down to the lower or upper edge of the agar plate to release the worms, about 4.5 cm away from each of the Sodium Azide drops (with either butanol or ethanol). If the worms remembered that food was marked with butanone, they will go to the butanone drop and get stuck there due to the Azide present there. If they do not remember, a similar number of animals will go to the ethanol drop as to the butanone drop. This also occurs if the animals are not butanone trained. After one hour of exploring the plates with butanone and ethanol, memory is quantified by the chemotaxis index. The chemotaxis index is calculated as the number of animals in the butanone spot minus the number of animals in the ethanol spot divided by the total number of animals. FIG. 5 shows the results of the memory test, wherein memory is expressed as chemotaxis index in view of the respective treatments in CL2122 and CL2355 animals.

### CONCLUSION

The control experiments as performed show that Metformin and AP do not increase the normal chemotaxis index of trained or untrained CL2122 animals. This indicates that normal memory of control animals was not affected by any treatment. In CL2355, the chemotaxis index was close to zero for butanone trained animals. This shows that the memory of these animals was almost entirely lost. Treatment of worms with AP at 111, 333 and 1000 IU/drop dose dependently, and statistically significantly increased the memory of CL2355 animals compared to buffer treated controls. Also, the metformin treated animals showed a statistically significant increase in memory in this test. Therefore, treatment with AP counteracts the loss of memory in a dose specific manner. The effect of AP to counteract memory loss is somewhat less than that of metformin which is known to act against neurodegeneration. This difference is likely due to the fact that the worm absorbs the metformin within 48 hours, whereas the AP is not and will be cleared in a matter of minutes. After treatment with test substances (AP or metformin), the entire learning process and memory test part takes place in the absence of these test substance. This test time is about 3 hours and during this period metformin may last longer because it is likely to be absorbed into the body and may provide a longer lasting protective effect in comparison to AP.

1. An alkaline phosphatase for use in the treatment or prophylaxis of a mammal suffering from or at risk of a neurodegenerative disorder caused by a reduction of Peroxisome proliferator-activated receptor gamma coactivator 1 (PGC1) activity as compared to a healthy individual, wherein said treatment comprises administering to said mammal a therapeutically effective amount of alkaline phosphatase that inhibits neurodeg by preventing reduction of PGC1 activity.

2. The alkaline phosphatase for use according to claim 1, wherein the neurodegenerative disorder is selected from the group consisting of Alzheimer's Disease, frailty related disorders, Parkinson's Disease, Amyotrophic Lateral Sclerosis, Multiple Sclerosis and neurodegeneration as a result of stroke.

3. The alkaline phosphatase for use according to claim 1, wherein said preventing reduction of PGC1 activity comprises an increase in the activation of adenosine monophosphate-activated protein kinase (AMPK) by alkaline phosphatase to promote an anti-stress response.

4. The alkaline phosphatase for use according to claim 1, wherein said therapeutically effective amount is 6 to 1350 U/day/kg alkaline phosphatase.

5. The alkaline phosphatase for use according to claim 1, wherein the alkaline phosphatase is a tissue specific ecto-phosphatase selected from the group consisting of intestinal AP (IAP), placental ALP (PALP) and liver AP (LAP.

6. The alkaline phosphatase for use according to claim 1, wherein said treatment comprises intravenous, parenteral or oral administration.

7. The alkaline phosphatase for use according to claim 1, wherein the alkaline phosphatase is a recombinant alkaline phosphatase.

8. The alkaline phosphatase for use according to claim 1, wherein the alkaline phosphatase is one or more selected from the group consisting of a biological active fragment or derivative of alkaline phosphatase, synthetic alkaline phosphatase derivative, and a small chemo-pharmaceutical molecule exerting functional alkaline phosphatase activity.

9. The alkaline phosphatase for use according to claim 1, wherein said treatment or prophylaxis comprises the delay of onset, or attenuated progression or prevention of progression of said neurodegenerative disorder.

10. The alkaline phosphatase for use according to claim 1, wherein said treatment comprises attenuation of the inflammatory response of a mammal suffering neurodegenerative disorder.

11. A method for inhibiting neurodegradation by preventing reduction of PGC1 activity of a mammal comprising administering to said mammal a therapeutically effective amount of an alkaline phosphatase according to claim 1.

12. A use of the alkaline phosphatase according to claim 1 for the preparation of a medicament for the prophylaxis of a mammal at risk of a neurodegenerative disorder caused by a reduction of PGC1 activity as compared to a healthy individual.

13. The alkaline phosphatase for use according to claim 2, wherein the neurodegenerative disorder is Parkinson's Disease, Alzheimer's Disease or frailty related disorders.

14. The alkaline phosphatase for use according to claim 4, wherein said therapeutically effective amount is 25 to 750 U/day/kg or 50 to 500 U/day/kg alkaline phosphatase.

15. The alkaline phosphatase for use according to claim 5, wherein the alkaline phosphatase is a tissue specific ecto-phosphatase selected from the group consisting of IAP and PALP.

16. The alkaline phosphatase for use according to claim 6, wherein said treatment comprises oral administration.

17. The alkaline phosphatase for use according to claim 7, wherein the alkaline phosphatase is a recombinant mammalian alkaline phosphatase or a human recombinant alkaline phosphatase.

18. The alkaline phosphatase for use according to claim 8, wherein the small chemo-pharmaceutical molecule exerting functional alkaline phosphatase activity is a biological active fragment or derivative of alkaline phosphatase.
